(19) United States
(12) Patent Application Publication
(10) Pub. No.: US 2014/0073619 A1
(43) Pub. Date: Mar. 13, 2014

(54) METHOD FOR TREATING CHRONIC PAIN

(71) Applicant: Southern Illinois University Edwardsville, Edwardsville, IL (US)

(72) Inventors: William L. Neumann, Edwardsville, IL (US); Daniela Salvemini, Edwardsville, IL (US); Smita Rausaria, Edwardsville, IL (US)

(73) Assignee: Southern Illinois University Edwardsville, Edwardsville, IL (US)

(21) Appl. No.: 14/077,650

(22) Filed: Nov. 12, 2013

Related U.S. Application Data
(62) Division of application No. 13/227,842, filed on Sep. 8, 2011.
(60) Provisional application No. 61/380,856, filed on Sep. 8, 2010.

(51) Int. Cl. A61K 9/00; A61K 45/06; A61K 31/555

(52) U.S. Cl. CPC A61K 9/0053; A61K 31/555; A61K 45/06
USPC 514/185

(57) ABSTRACT

The present invention provides analgesic compounds comprising at least one modified metalloporphyrin compound. Also provided are methods of treating pain by orally administering an analgesic compounds comprising at least one modified metalloporphyrin compound.

### CROSS REFERENCE TO RELATED APPLICATIONS

This is a non-provisional patent application and claims priority to U.S. Provisional Patent Application Ser. No. 61/380,856, filed on Sep. 8, 2010, and U.S. Non-provisional patent application Ser. No. 13/227,842, filed on Sep. 8, 2011, which are herein incorporated by reference in their entirety.

### FIELD

The invention generally relates to analgesic compounds comprising at least one modified metalloporphyrin compound. In particular, the invention relates to the administration of an orally available modified metalloporphyrin compound for the treatment of pain.

### BACKGROUND

Traditional multifaceted drug regimens for controlling chronic pain associated with inflammatory disorders such as arthritis may be marginally effective and produce highly variable results. Existing analgesic therapeutic compounds such as NSAIDs and COX-2 inhibitors may induce undesired gastrointestinal or cardiovascular side-effects. These existing analgesic compounds typically modify biochemical pathways that are down-stream from a key proinflammatory and neurotoxic biochemical pathway involved in transitioning from acute to chronic pain and in the maintenance of inflammatory pain. One key compound in this biochemical pathway may be peroxynitrite (PN), a modulator of inflammatory and chronic neuropathic pain. For example, the development of morphine-induced hyperalgesia and antinociceptive tolerance has been associated with PN overproduction. Molecules capable of directly scavenging or reducing PN concentrations may provide a novel and broadly effective analgesic and anti-inflammatory strategy.

One class of existing compounds that has shown promise at modulating PN-associated nociceptive pathways are the group of peroxynitrite decomposition catalysts (PNDCs) based on modified metalloporphyrin structures. An example of a typical metalloporphyrin structure is illustrated in Formula (I):

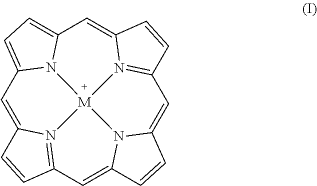

Because most naturally occurring metalloporphyrin compounds function as redox centers in biological systems, metalloporphyrin compounds are known to be highly potent redox catalysts, making them particularly well suited as a PNDC compound. However, the naturally occurring metalloporphyrin compounds are either encased in proteins such as cytochromes, or only exist for a brief period prior to catabolism. As a result, significant modifications to the naturally occurring metalloporphyrin compounds are required in order to enhance the solubility, stability, and other desired pharmaceutical properties in order to develop an effective PNDC compound.

One class of compounds that has shown promise at modulating PN-associated nociceptive pathways are peroxynitrite decomposition catalysts (PNDCs) based on metalloporphyrin structures with attached multiply-charged ligand systems, such as the meso-N-alkylpryridinium compound Mn(III)TMPyP⁵⁺ shown in Formula (II) below:

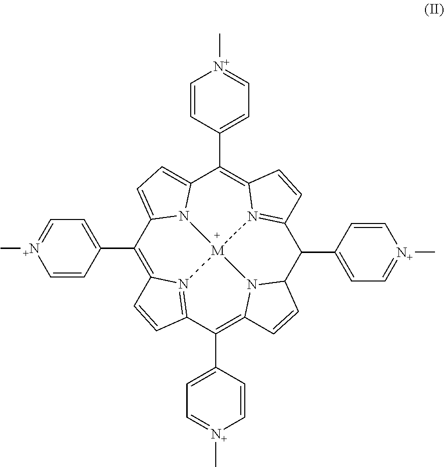

These existing metalloporphyrin-based PNDC compounds are typically highly polar and highly water soluble as a result of the multiple attached charged ligands. Since the charge-carrying meso-substituted ligand systems of these compounds are typically electron-withdrawing, these compounds also possess relatively high reduction potentials, resulting in substantial superoxide dismutase-mimic (SODm) activity. However, these compounds are generally not orally active, nor do they readily penetrate the blood-brain barrier (BBB), posing significant challenges for the use of these compounds for the long-term treatment of chronic pain.

Attempts to enhance the lipid solubility of existing metalloporphyrin-based PNDC compounds have involved flexible hydrophobic substitutions within the attached ligands, such as a TnHex-2-PyP ligand, shown in Formula (III).

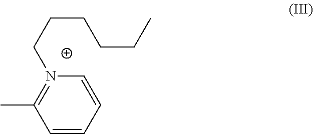

Although these flexible hydrophobic substitutions have resulted in improved in vivo potency due to enhanced membrane penetration properties, the oral efficacy of these compounds remains undocumented. Further, these lipophilic compounds may be toxic due to unfavorable ion channel binding and mitochondrial sequestration activities.

A need exists for an orally available PNDC compound with comparable effectiveness to existing metalloporphyrin-based complexes, as well as sufficiently effective membrane penetration properties for crossing the blood-brain barrier. Such a compound would make possible the ongoing treatment of neuropathic and chronic pain disorders by targeting the modulation of peroxynitrite-related nociceptive pathways using oral administration methods.

### SUMMARY

Additional objectives, advantages and novel features will be set forth in the description which follows or will become apparent to those skilled in the art upon examination of the drawings and detailed description which follows.

### BRIEF DESCRIPTION OF THE DRAWINGS

FIG. 1 is a graph summarizing the inhibition of TNF-α production by the BV2 cell cultures treated with different concentrations of a PNDC compound 3 hours after LPS stimulation.

FIG. 2 is a graph summarizing the inhibition of carrageenan-induced thermal hyperalgesia in rats injected with different concentrations of PNDC compounds for 5 hours after a carrageenan injection.

FIG. 3 is a graph summarizing the inhibition of carrageenan-induced thermal hyperalgesia in rats injected with different concentrations of PNDC compounds for 5 hours after a carrageenan injection.

FIG. 4 is a graph summarizing the inhibition of carrageenan-induced thermal hyperalgesia in rats orally administered a PNDC compound for a period of 5 hours after carrageenan injection.

FIG. 5 is a graph summarizing the inhibition of carrageenan-induced thermal hyperalgesia in rats orally administered a PNDC compound for a period of 5 hours after carrageenan injection compared with rats injected with a different PNDC compound.

FIG. 6 is a graph summarizing the inhibition of taxol-induced thermal hyperalgesia in mice orally administered a PNDC compound for a period of 3 hours after the commencement of the PNDC treatment.

Corresponding reference characters and labels indicate corresponding elements among the view of the drawings. The headings used in the figures should not be interpreted to limit the scope of the claims.

### DETAILED DESCRIPTION

Orally available peroxynitrite decomposition catalyst (PNDC) compounds are provided that are based on novel modified porphyrin structures. The modifications to the porphyrin structures render the compounds orally available and capable of crossing the blood-brain barrier (BBB), while retaining high PNDC efficacy. Examples of orally available PNDC compounds, methods of producing the PNDC compounds, and methods of using the PNDC compounds to treat chronic pain associated with neuropathic, inflammatory, or other disorders are described in detail below.

### I. Orally Available PNDC Compounds

The orally available PNDC compounds are capable of reacting directly with PN and of catalyzing PN decomposition. In addition, these PNDC compounds may also inhibit the biosynthesis of PN by by functioning as superoxide dismutase mimics (SODm) and removing superoxide compounds. By directly decomposing and/or preventing the formation of PN, treatment with the orally available PNDC compounds may result in the inhibition of thermal, cold and tactile hypersensitivities associated with chronic neuropathic pain. In addition, these orally available PNDC compounds inhibit the development of inflammatory pain and synergize with the effects of known analgesic compounds including cyclooxygenase 2 (COX-2) inhibitors, non-selective NSAIDs, opiates and anti-metabolites such as methotrexate. Further, treatment with the orally available PNDC compounds may inhibit the development of opiate anti-nociceptive tolerance at doses well below the amount at which behavioral side effects occur.

The orally available PNDC compounds may include a variety of specific chemical structures including but not limited to tetracyclohexanoporphyrin (TCP) derivatives, hybrid porphyrinoid (HP) derivatives, octahydroacridine bis-salicylimine (OBS) derivatives, tetracyclohexyl corrole (TCC) derivatives, electron-rich porphyrin (ERP) derivatives and other porphyrin derivatives, all of which are described below.

a. Tetracylohexanoporphyrin (TCP) Derivatives

In one aspect, the orally available PNDC compounds may include tetracylohexanoporphyrin (TCP) derivatives, shown in Formula (IV):

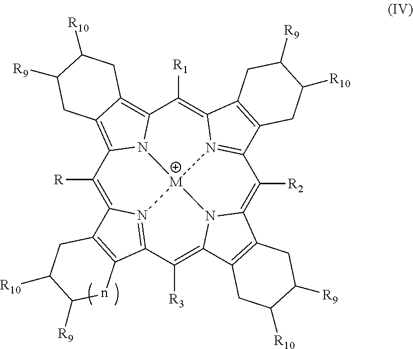

In formula (IV), R₁-R₁₀ are attached groups independently chosen from the ligands shown in Table I. The transition metal ion M may be chosen from Mn, Fe, Ni, Co, Cu, Zn, and V ions. Alternatively, the transition metal ion M may be optionally excluded in the TCP derivative compound.

TABLE 1
TCP Derivative Attached Groups
R₁-R₈ Groups

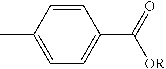

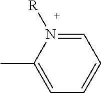

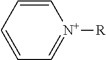

—NO₂
—CF₃
—CO₂R
—H
R₉-R₁₀ Groups
—OH
—CH₂OR
—CH₂OH
—CO₂R
—H

In Table 1, R may be chosen from H, alkanes, alkenes, alkynes, carboxyalkanes, halogens including Br, Cl, and F, nitrogen-containing groups including NO₂, NH₂, CONH₂, NHCO(R₁₁) where R₁₁ may be hydrogen, alkyl, or alkylaryl, N(R₁₁)₂ where each R₁₁ may be chosen independently from the R₁₁ group described previously, SeR₁₁, TeR₁₁, sulfur-containing groups including SO₃H, SR₁₁, CF₃, OR₁₁ including OH, substituted alkenes including pyridyls, and combinations thereof. The meso substituents R₁-R₄ may be all identical groups from Table 1, all non-identical groups from Table 1, or a portion of the meso substituents R₁-R₄ may be identical and another portion may be non-identical groups from Table 1.

The TCP derivative compounds may be complexed with a transition metal ion M⁺, as shown in Formula (IV). The TCP derivative compounds incorporate cyclohexyl groups dispersed symmetrically around the periphery of the central porphyrin structure that create local zones of lipophilicity, resulting in a global reduction of the polar surface area of the TCP derivatives. Because these zones of lipophilicity are situated on the periphery of the TCP derivative's chemical structure, these cyclohexyl groups significantly enhance the ability of the TCP derivatives to penetrate membranes. As a result, the TCP derivatives may penetrate membranes, including the endothelial cell membranes of the BBB, in a manner similar to that of ionophore-like compounds such as valinomycin.

In addition, the oral bioavailability of the TCP derivatives may be enhanced as a result of increased molecular rigidity due to the added cyclohexyl groups. Molecular rigidity, which may be estimated by the number of rotatable bonds within a chemical structure, is thought to be a significant factor governing the oral availability of a compound.

Exemplary TCP derivatives are illustrated in Table 2 below:

TABLE 2
Exemplary TCP Derivatives

| No. | Structure |
|---|---|
| TCP-1 | 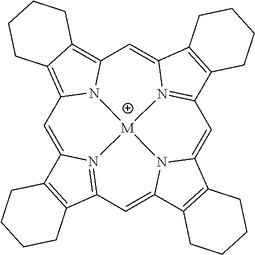 |
| TCP-2 | 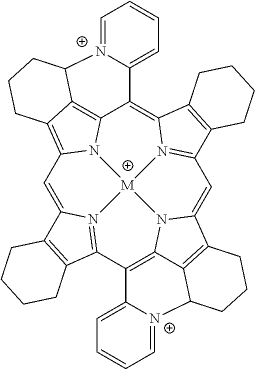 |
| TCP-3 | 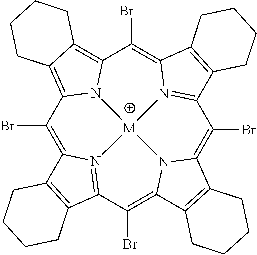 |
| TCP-4 | 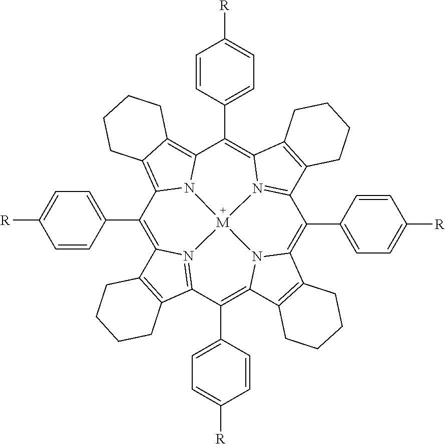 |
| TCP-5 | 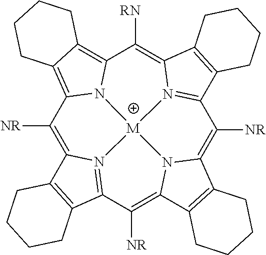 |
| TCP-6 | 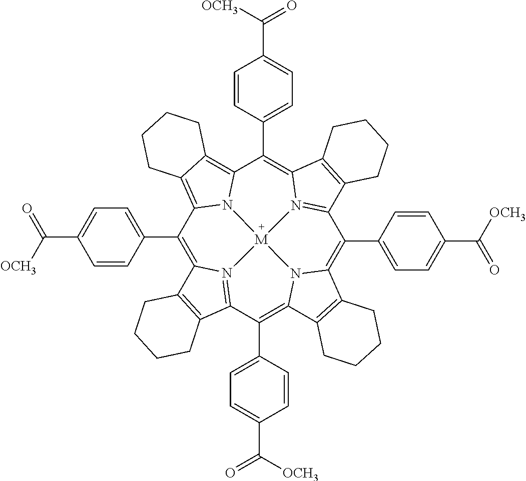 |
| TCP-7 | 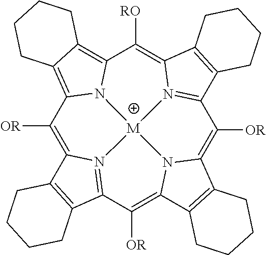 |
| TCP-8 | 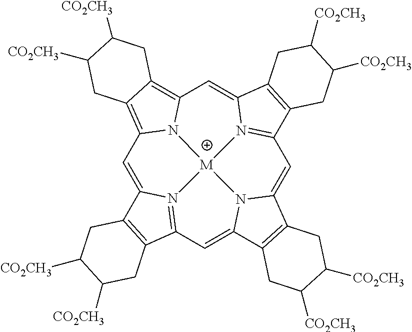 |
| TCP-9 | 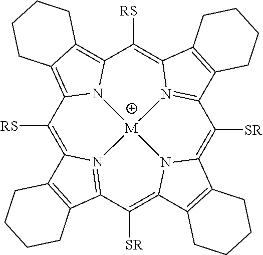 |
| TCP-10 | 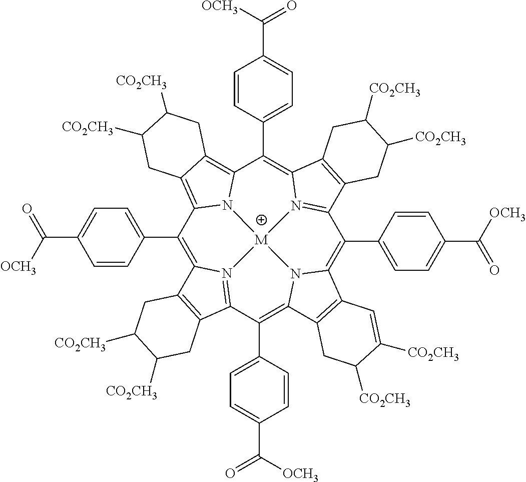 |
| TCP-11 | 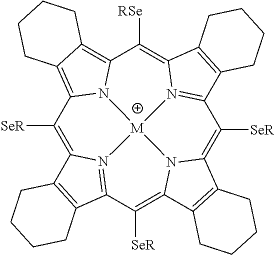 |
| TCP-12 | 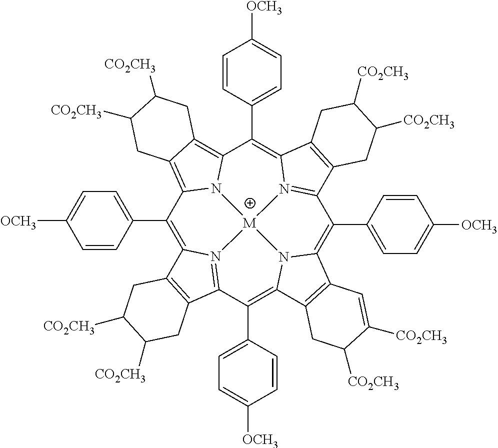 |
| TCP-13 | 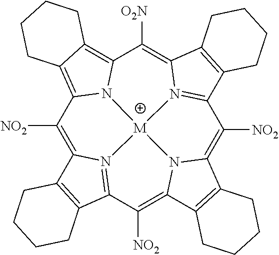 |
| TCP-14 | 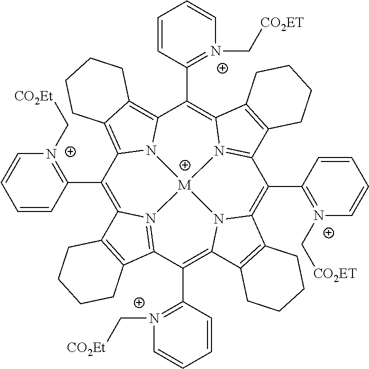 |
| TCP-15 | 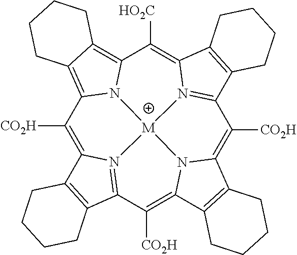 |
| TCP-16 | 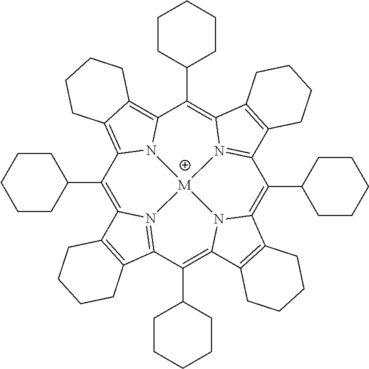 |
| TCP-17 | 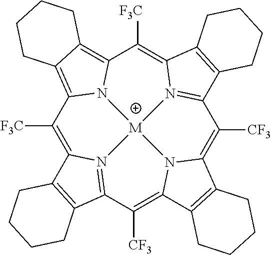 |
| TCP-18 | 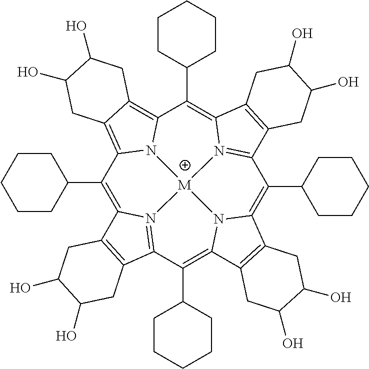 |
| TCP-19 | 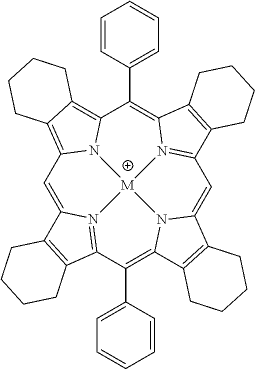 |
| TCP-20 | 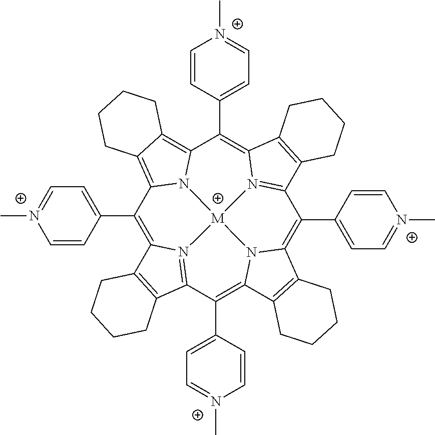 |
| TCP-21 | 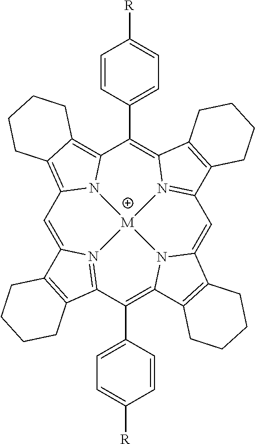 |
| TCP-22 | 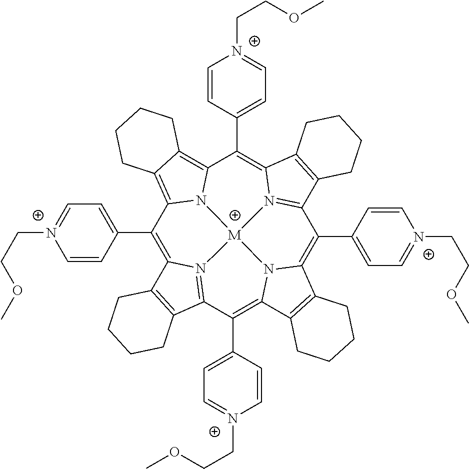 |
| TCP-23 | 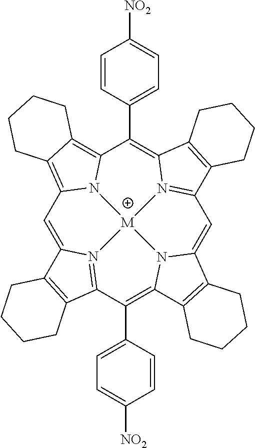 |
| TCP-24 | 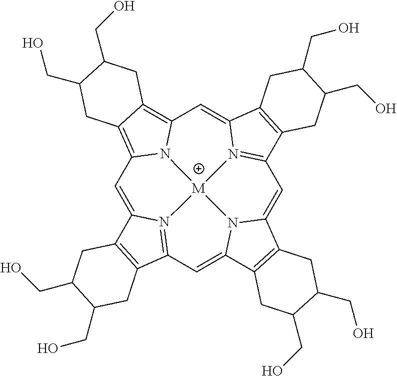 |
| TCP-25 | 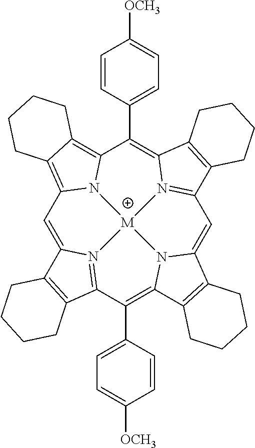 |
| TCP-26 | 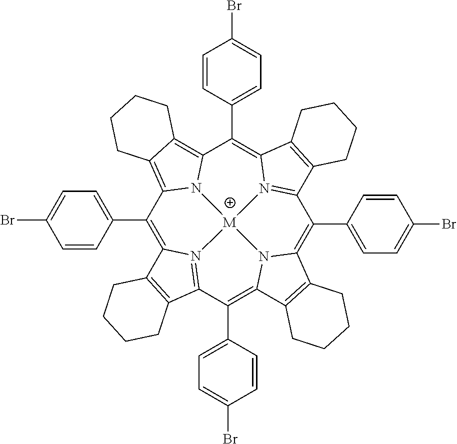 |
| TCP-27 | 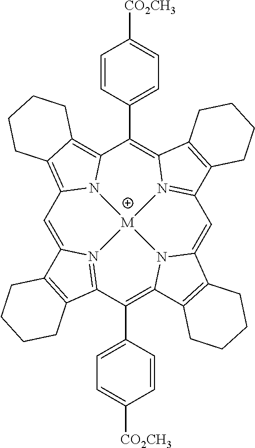 |
| TCP-28 | 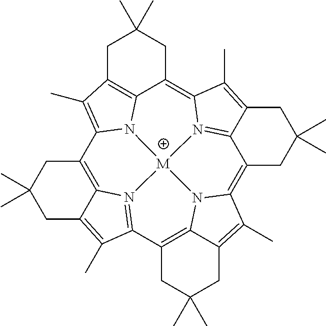 |
| TCP-29 | 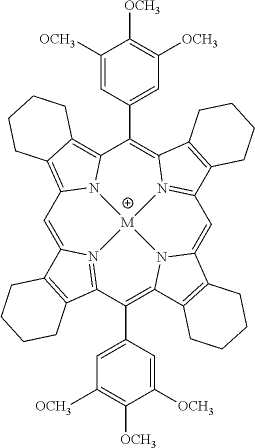 |
| TCP-30 | 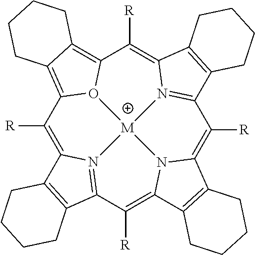 |
| TCP-31 | 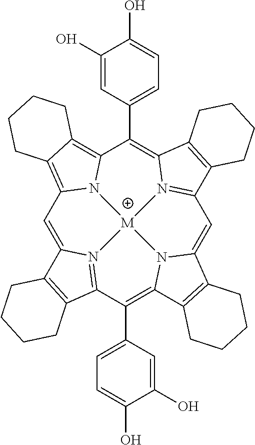 |
| TCP-32 | 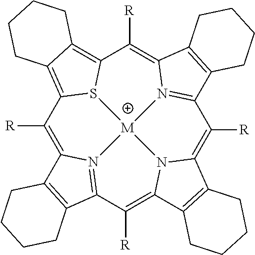 |
| TCP-33 | 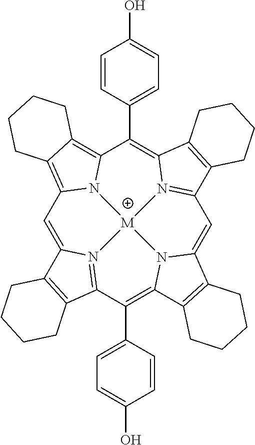 |
| TCP-34 | 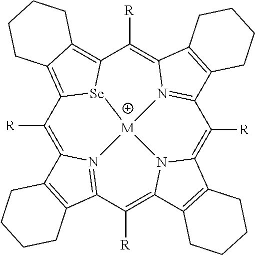 |
| TCP-35 | 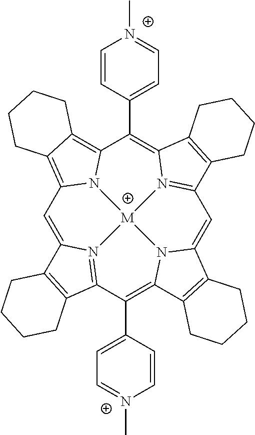 |

The R groups illustrated in Table 2 may be any of the same compounds from the R groups described above for Table 1.

b. Hybrid Porphyrinoid (HP) Derivatives

In another aspect, the orally available PNDC compounds may include hybrid porphyrinoid (HP) derivatives, shown in Formula (V):

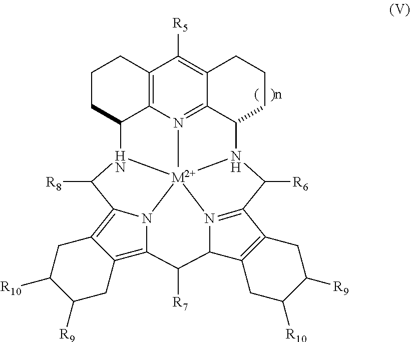

In formula (V), R₅-R₁₀ are attached groups independently chosen from the ligands shown in Table I above. The transition metal ion M²⁺ may be chosen from Mn, Fe, Ni, Co, Cu, Zn, and V ions. Alternatively, the transition metal ion M²⁺ may be optionally excluded in the HP derivative compound.

The HP derivative compounds employ a octahydroacridine scaffold structure rather than the central porphyrin structure employed in the TCP derivative compounds, and as a result may differ functionally from the TCP derivative compounds. For example, the HP derivatives include a monoanionic ligand set rather than the porphyrin dianionic ligand; this difference in ligand may directly influence the mechanism and type of catalysis resulting from the HP derivatives including but not limited to activity toward PN, SO, and H₂O₂. In addition, the R₅ ligand is in direct conjugation with the metal center of the HP derivative compounds (see Formula IV), and thus the catalysis properties of the HP derivative compounds may be sensitive to the particular properties of the R₅ ligand due to the electronic push-pull effects acting through the octahydroacridine's pyridine group.

c. Octahydroacridine bis-Salicylimine (OBS) Derivatives

In yet another aspect, the orally available PNDC compounds may include octahydroacridine bis-salicylimine (OBS) derivatives, shown in Formula (VI):

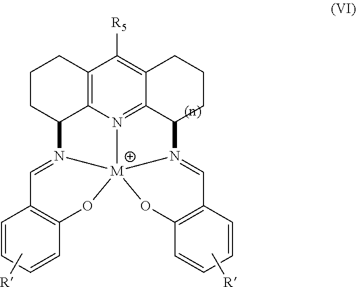

In formula (VI), R₅ is an attached group chosen from the ligands shown in Table I above, and R represents a substitution of any of the carbons in the salicylimine rings, wherein the substitute atom may include, but is not limited to N, O, S, and Se. The transition metal ion M⁺ may be chosen from Mn, Fe, Ni, Co, Cu, Zn, and V ions. Alternatively, the transition metal ion M⁺ may be optionally excluded in the TCP derivative compound.

The OBS derivatives employ the octahydroacridine as an acyclic bis-salicylimine ligand scaffold, resulting in a relatively rigid acyclic ligand system with sufficient stability for enhanced oral availability. Like the HP derivatives, the catalysis activity of the OBS derivatives may be relatively sensitive to the particular chemical properties of the R₅ ligand group, due to the electronic push-pull effects acting through the octahydroacridine's pyridine group.

d. Tetracyclohexyl Corrole (TCC) Derivatives

In still yet another aspect, the orally available PNDC compounds may include tetracyclohexyl corrole (TCC) derivative compounds. Corroles are defined herein as tetrapyrrole macrocycles that are closely related to porphyrins in structure, with one less carbon atom in the outer periphery and an additional NH proton in the inner core. Although existing corrole derivatives are known to be pharmaceutically active compounds, these existing corrole derivative compounds face similar limitations in oral availability and membrane penetration properties to existing metalloporphyrin-based PNDC compounds, such as the compound shown in Formula (II). An example of a tetracyclohexyl corrole (TCC) derivative is shown in Formula (VII):

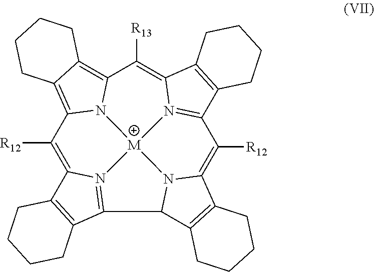

The R₁₂ and R₁₃ attached groups in Formula (VII) may be chosen independently from the ligands shown in Table 3 below. The TCC derivative compounds may be optionally complexed with a transition metal ion M⁺ chosen from Mn, Fe, Ni, Co, Cu, Zn, and V ions.

TABLE 3
TCC Derivative Attached Groups
R₁₂-R₁₃ Groups

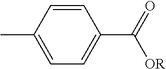

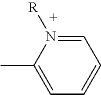

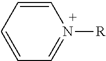

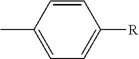

—CF₃
—CO₂R
—H
—NO₂
—OH
—CH₂OR
—CH₂OH

In Table 3, R may be chosen from the same group described for Table 1, including but not limited to: H, alkanes, alkenes, alkynes, carboxyalkanes, halogens including Br, Cl, and F, nitrogen-containing groups including NO₂, NH₂, CONH₂, NHCO(R₁₁) where R₁₁ may be hydrogen, alkyl, or alkylaryl, N(R₁₁)₂ where each R₁₁ may be chosen independently from the R₁₁ group described previously, SeR₁₁,TeR₁₁, sulfur-containing groups including SO₃H, SR₁₁, CF₃, OR₁₁ including OH, substituted alkenes including pyridyls, and combinations thereof. The substituents R₁₂ may be identical groups from Table 3 or non-identical groups from Table 3.

The lipophilic cyclohexyl groups dispersed symmetrically around the periphery of the central corrole structure create local zones of lipophilicity, significantly enhancing the ability of the TCC derivatives to penetrate membranes. As a result, the TCC derivatives are capable of crossing the BBB in a manner similar to the TCP derivatives. The TCC compounds may be administered as a TCC derivative as shown in Formula (VI) above, or the administered TCC compounds may be complexed with a transition metal ion M⁺ including but not limited to Mn, Fe, Ni, Co, Cu, Zn, and V ions, as illustrated in compound TCC-3 in Table 4 below.

Exemplary TCC derivative compounds are illustrated in Table 4:

TABLE 4
Exemplary TCC Derivatives

| No. | Structure |
|---|---|
| TCC-1 | 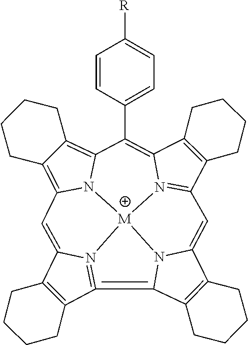 |
| TCC-2 | 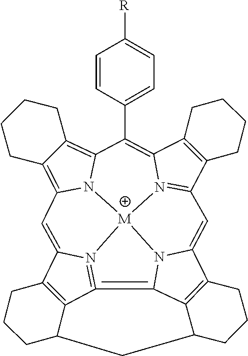 |
| TCC-3 | 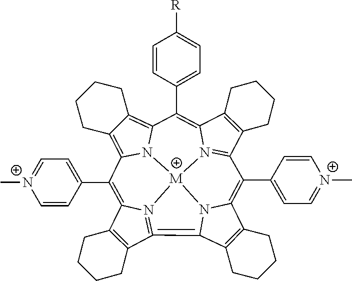 |

In Table 4, R may be chosen from the same group described for Table 3 above.

e. Electron-Rich Porphyrin (ERP) Derivatives and Other Porphyrin (OP)

Derivatives

In an additional aspect, the orally available PNDC compounds may include electron-rich porphyrin (ERP) derivatives as shown in Formula (VIII):

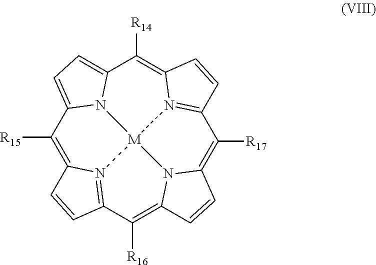

The R₁₄-R₁₇ attached groups shown in Formula (VIII) may be chosen independently from any ligand including but not limited to the attached groups shown in Table 5. Alternatively, the transition metal ion M may be optionally excluded in the TCP derivative compound.

TABLE 5
ERP Derivative Attached Groups
R₁₄-R₁₇ Groups

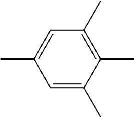

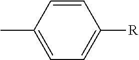

—OR
—NHR
—SR
—CO₂R
—SeR
—TeR
—R

In Table 5, R may be chosen from the same group described for Table 1, including but not limited to: H, alkanes, alkenes, alkynes, carboxyalkanes, halogens including Br, Cl, and F, nitrogen-containing groups including NO₂, NH₂, CONH₂, NHCO(R₁₁) where R₁₁ may be hydrogen, alkyl, or alkylaryl, N(R₁₁)₂ where each R₁₁ may be chosen independently from the R₁₁ group described previously, SeR₁₁,TeR₁₁, sulfur-containing groups including SO₃H, SR₁₁, CF₃, OR₁₁ including OH, substituted alkenes including pyridyls, and combinations thereof. Any two or more of the substituents R₁₄-R₁₇ may be identical groups from Table 5. Alternatively, substituents R₁₄-R₁₇ may be non-identical groups from Table 5, or any combination of identical and non-identical groups from Table 5.

Exemplary ERP derivative compounds are illustrated in Table 6:

TABLE 6
Exemplary ERP Derivatives

| No. | Structure |
|---|---|
| ERP-1 | 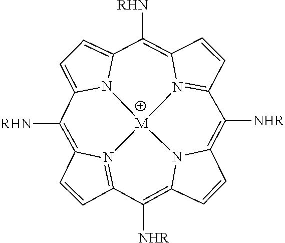 |
| ERP-2 | 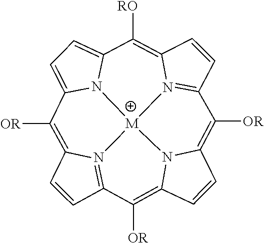 |
| ERP-3 | 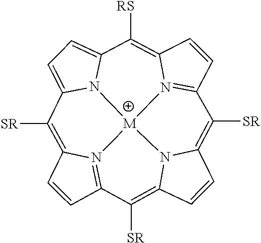 |
| ERP-4 | 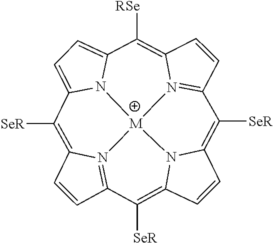 |
| ERP-5 | 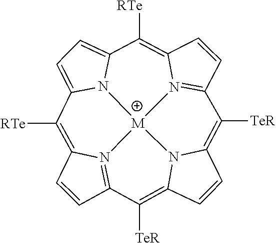 |

In Table 6, R may be chosen from the R groups defined for Table 5 above. The transition metal ion M may be chosen from Mn, Fe, Ni, Co, Cu, Zn, and V ions.

In another additional aspect, the orally available PNDC compounds may include other porphyrin (OP) derivative compounds, shown in Table 7:

TABLE 7
Exemplary Other Porphyrin (OP) Derivatives

| No. | Structure |
|---|---|
| OP-1 | 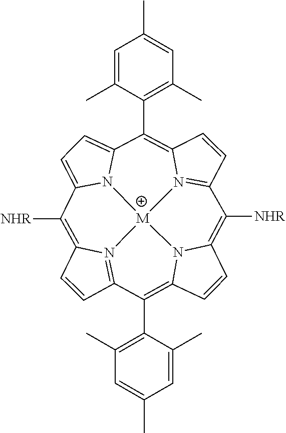 |
| OP-2 | 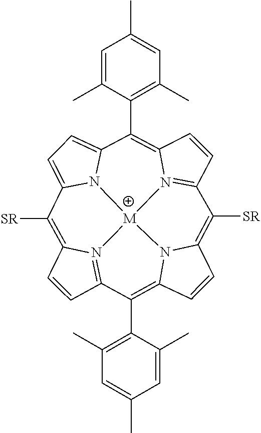 |
| OP-3 | 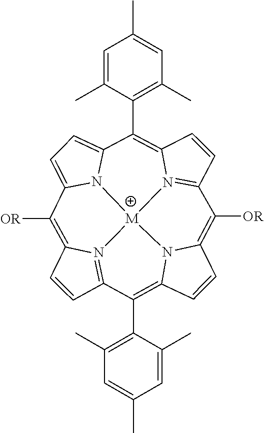 |
| OP-4 | 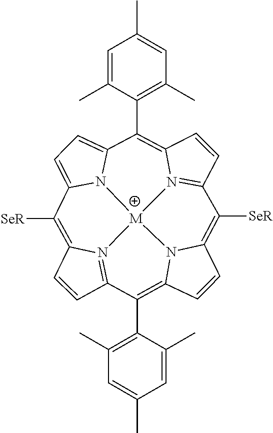 |
| OP-5 | 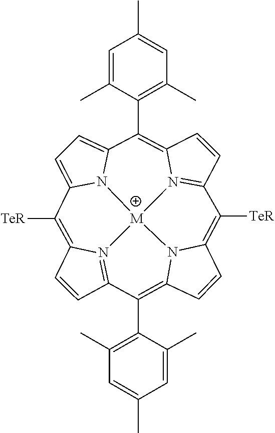 |
| OP-6 | 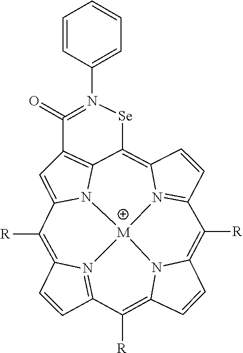 |
| OP-7 | 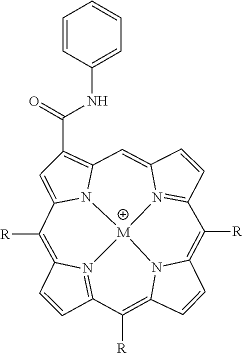 |
| OP-8 | 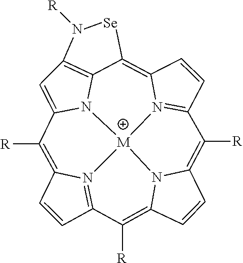 |
| OP-9 | 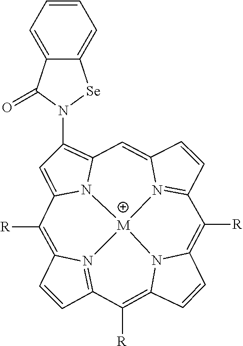 |
| OP-10 | 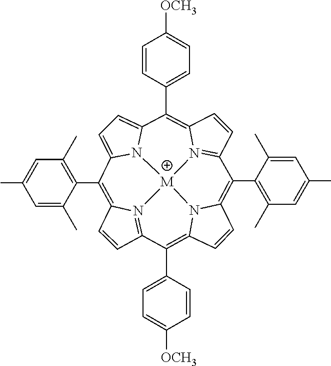 |
| OP-11 | 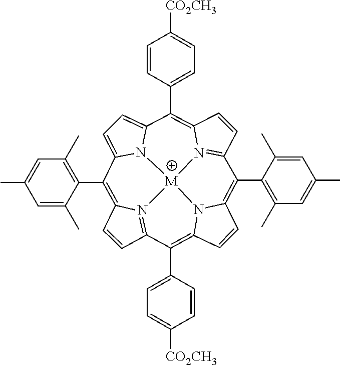 |

In Table 7, R may be chosen from the R groups defined for Table 5 above.

### II. Methods of Producing Orally Available PNDC Compounds

The methods of producing the orally available PNDC may be adopted and modified from any known synthetic methods including but not limited to modifications of processes used in the synthesis of optical dyes. Exemplary methods of producing the orally available PNDC compounds are described in detail below.

a. Method of Producing Tetracylohexanoporphyrin (TCP)

TCP may be prepared by a modification of an existing synthesis procedure (Ono 1988). An example of a modified synthesis method for producing TCP is illustrated below:

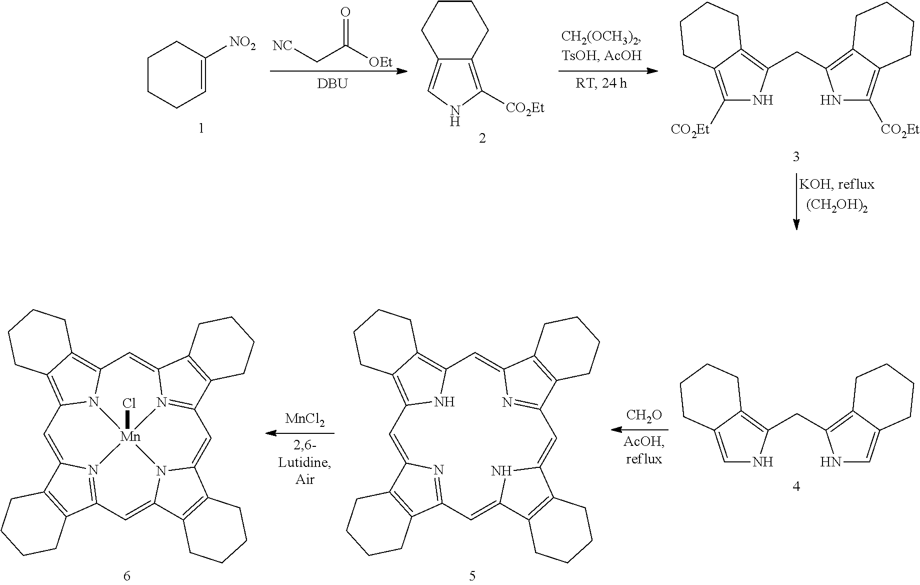

In this method, 1-nitrocyclohexene 1 may be converted to the isoindole 2 via the Barton-Zard reaction (Ono 2008). Compound 2 may be converted to dipyrrylethane-diester 3 using previously reported methods (Filatov et al. 2008). The resulting dipyrrylethane-diester 3 may be deprotected to dipyrrylmethane 4 by a slight modification of an existing method (Lash 1992). In a key step, dipyrrylmethane 4 may be converted to the TCP compound 5. The resulting TCP ligand 5 may be subjected to metallation with MnCl₂ using 2,6-lutidine as base and transfer ligand, resulting in the TCP PNDC compound. Various other ligands described above may be attached to this basic TCP compound to produce other TCP derivative compounds.

b. Method of Producing bis-meso-Substituted Tetracylohexanoporphyrin (TCP) Derivatives

The intermediate product dipyrrylmethane 4 from the TCP synthesis shown above may be used to produce a series of bis-meso-substituted TCP derivative compounds using the method illustrated below. Compound 4 may be reacted with benzaldehyde and methyl 4-formylbenzoate to produce bis-meso-phenyl-TCP 7 and bis-4-methoxycarbonyl-phenyl-TCP 8. The bis-meso-phenyl TCP 7 may be metallated to produce a diphenyl TCP derivative 9 that is similar to TCP-19 from Table 2 above. TCP 8 may be metallated as above to produce a bis-4-methoxycarbonyl-phenyl TCP derivative 10 that is similar to TCP-27 from Table 2 above.

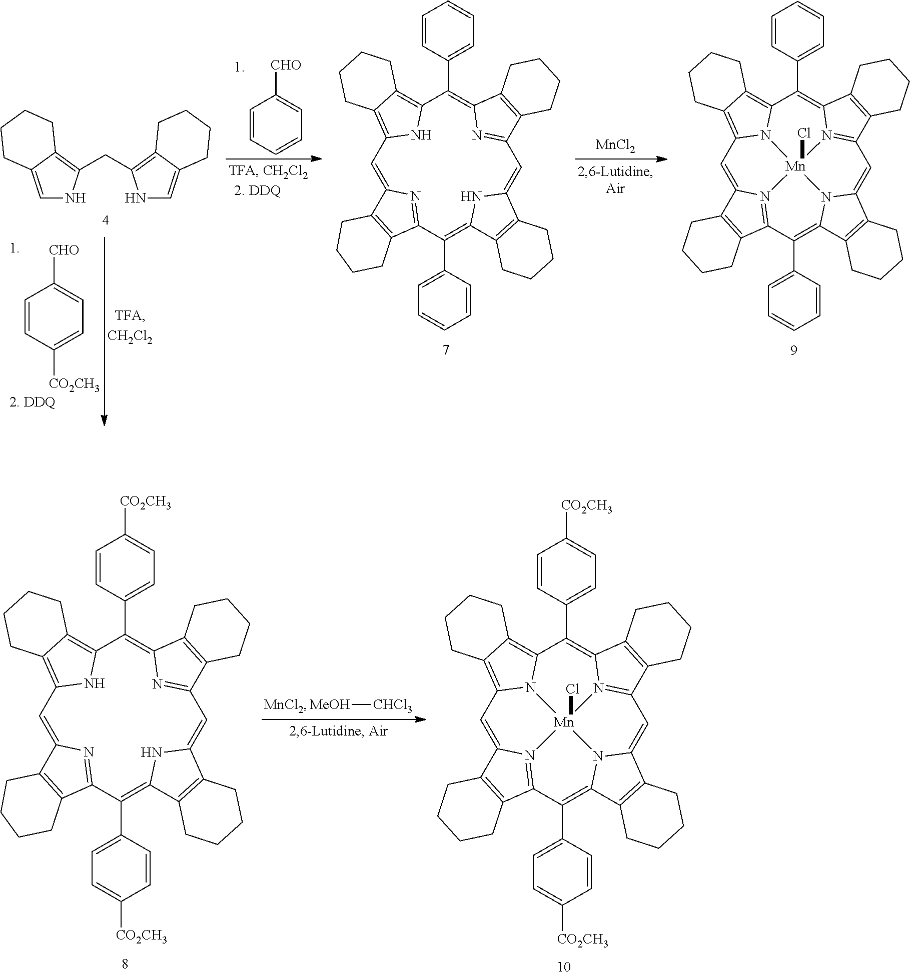

c. Method of Producing tetra-meso-Substituted Tetracylohexanoporphyrin (TCP) Derivatives

The intermediate isoindole ethyl ester 2 product from the method of producing TCP described above may be used to produce a tetra-meso-substituted TCP derivative compound using the synthesis method illustrated below:

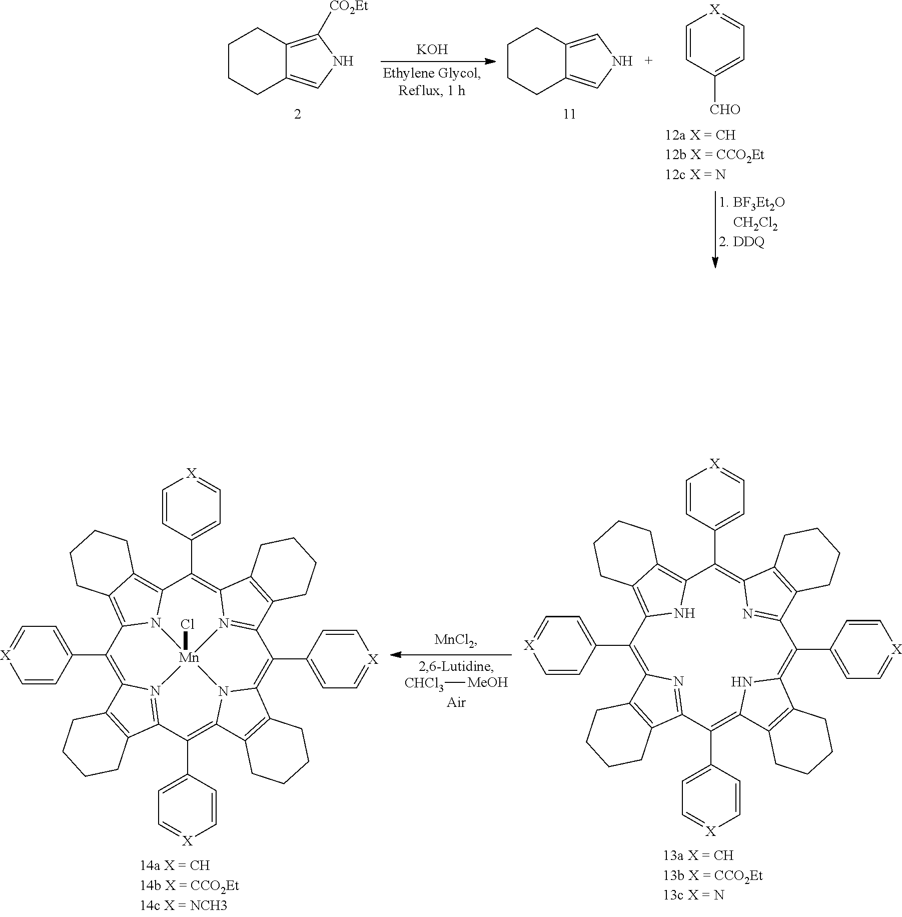

The isoindole ethyl ester 2 may be hydrolyzed and decarboxylated using a modification of an existing procedure (Lash 1992) to produce an unstable isoindole 11. Compound 11 may then be reacted with benzaldehyde 12a to produce tetra-meso-phenyl-TCP 13a. Alternatively, compound 11 may be reacted with methyl 4-formylbenzoate 12b to produce tetra-meso-4-carboxymethyl-TCP 13b, or compound 11 may be reacted with 4-pyridinecarboxaldhehyde 12c to produce tetra-4-meso-pyridyl-TCP 13c. Compounds 13a, 13b, and 13c may then be metallated to produce the corresponding tetra-meso-functionalized metal-charge-shielded PNDC compounds 14a, 14b, and 14c, respectively.

d. Method of Producing Octahydroacridine bis-Salicylimine Derivatives

The octahydroacridine scaffold may be used to produce octahydroacridine bis-salicylimine derivative PNDC compounds. A non-limiting exemplary method of producing the octahydroacridine bis-salicylimine derivative PNDC compounds is illustrated below:

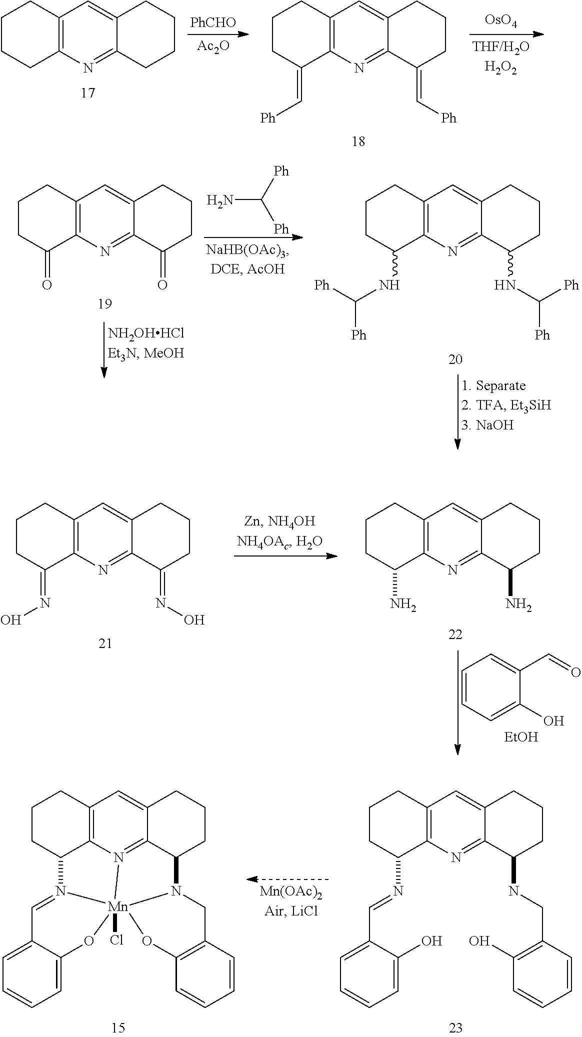

In this method, octahydroacridine 17 may be converted to a bis-benzylidine derivative 18 by reacting compound 17 with benzaldehyde in refluxing acetic anhydride. The olefins of compound 18 may then cleaved to produce acridine-dione 19 using catalytic osmium tetroxide and hydrogen peroxide as the secondary oxidant. Compound 19 may then be converted to a mixture of meso- and d,l-octahydroacridinediamines 20 and 21 by either of two procedures. In the first procedure, reductive amination with benzhydrylamine using sodium triacetoxyborohydride may be used to produce the diamines 20. In an alternative second procedure, the dione 19 may be converted to a bis-oxime 21, which crystallizes from solution. The desired isomers 20 may then be separated from solution by flash chromatography and the benzhydryl groups may be reductively removed with triethylsilane in refluxing TFA to produce, after free-basing, the desired d,l-diamine 22 as a mixture of isomers. The bis-oxime 21 may also be reduced with Zn^{o} to a mixture of diamine isomers, including diamine isomer 22. The d,l-diamine 22 may then be condensed with salicylaldehyde to form the desired bis-salicylimine compound 23, which may be further converted to a octahydroacridine bis-salicylimine derivative PNDC 15 by reaction with Mn(OAc)² and air oxidation.

### III. Methods of Using Orally Available PNDC Compounds

The orally available PNDC compounds described above may be included in a pharmaceutical composition that may be administered orally to a patient to treat a pain condition. The pain condition may be include an acute pain condition lasting less than about 6 months and a chronic pain condition lasting more than about 6 months. Acute pain is typically associated with a discrete injury or may be an intermittent brief episode of a longer-term disorder such as a migraine headache or a flare-up of rheumatoid arthritis-associated pain. A chronic pain condition may include a nociceptive condition caused by activation of nociceptors and a neuropathic condition caused by damage to or malfunction of the nervous system. Non-limiting examples of disorders that may be accompanied by chronic pain include arthritis such as rheumatoid arthritis, back pain, cancer, chronic fatigue syndrome, complex regional pain syndrome, restless leg syndrome, clinical depression, fibromyalgia, headache, sciatica, peripheral neuropathy, spinal stenosis, and idiopathic pain.

The pharmaceutical compositions that are orally administered may be manufactured in one or several dosage forms known in the art. Non-limiting examples of dosage forms include tablets such as suspension tablets, chewable tablets, effervescent tablets or caplets; pills; powders such as sterile packaged powders, dispensable powders, and effervescent powders; capsules including both soft or hard gelatin capsules such as HPMC capsules; lozenges; sachets; sprinkles; reconstitutable powders or shakes; troches; pellets; granules; liquids; suspensions; emulsions; semisolids; and gels.

The pharmaceutical compositions, in addition to being suitable for administration in multiple dosage forms, may also be administered with various dosage regimens. It is contemplated that the ingredients forming the various pharmaceutical compositions of the invention may be formulated into the same dosage form or in separate dosage forms and included in a variety of packaging options. The dosage forms may also be bi-daily, weekly, bi-weekly, monthly, or bi-monthly dosages of any of the ingredients. Typically, the dosage form will provide a daily dosage. The different dosage forms may be packaged separately or they may in be included within the same package contained in different cavities, such as in a strip pack or a blister pack.

The pharmaceutical compositions may include one or more of the orally available PNDC compounds described above, as well as one or more excipients. Non-limiting examples of suitable excipients include binders, fillers, non-effervescent disintegrants, effervescent disintegrants, preservatives, diluents, flavor-modifying agents, sweeteners, lubricants, dispersants, color additives, taste-masking agents, pH modifiers, and combinations thereof.

Non-limiting examples of binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof. The polypeptide may be any arrangement of amino acids ranging from about 100 to about 300,000 daltons.

Non-limiting examples of suitable fillers include carbohydrates, inorganic compounds, and polyvinilpirrolydone. For example, the filler may be calcium sulfate, both di-and tri-basic, starch, calcium carbonate, magnesium carbonate, microcrystalline cellulose, dibasic calcium phosphate, tricalcium phosphate, magnesium carbonate, magnesium oxide, calcium silicate, talc, modified starches, lactose, sucrose, mannitol, and sorbitol.

Non-limiting examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth.

Non-limiting examples of effervescent disintegrants include sodium bicarbonate in combination with citric acid and sodium bicarbonate in combination with tartaric acid.

Non-limiting examples of preservatives include antioxidants, such as a-tocopherol or ascorbate, and antimicrobials, such as parabens, chlorobutanol or phenol. Non-limiting examples of diluents include pharmaceutically acceptable saccharides such as sucrose, dextrose, lactose, microcrystalline cellulose, fructose, xylitol, and sorbitol; polyhydric alcohols; a starch; pre-manufactured direct compression diluents; and mixtures of any of the foregoing.

Non-limiting examples of flavor-modifying agents includes synthetic flavor oils and flavoring aromatics and/or natural oils, extracts from plants, leaves, flowers, fruits, and combinations thereof. Natural oils may include cinnamon oils, oil of wintergreen, peppermint oils, clover oil, hay oil, anise oil, eucalyptus, vanilla, citrus oil, such as lemon oil, orange oil, grape and grapefruit oil, and fruit essences including apple, peach, pear, strawberry, raspberry, cherry, plum, pineapple, and apricot.

Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; “Stevia rebaudiana” (Stevioside); chloro derivatives of sucrose such as sucralose; sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

Suitable non-limiting examples of lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

Non-limiting examples of suitable color additives include food, drug and cosmetic colors (FD&C), drug and cosmetic colors (D&C), and external drug and cosmetic colors (Ext. D&C). These colors or dyes, along with their corresponding lakes, and certain natural and derived colorants may be suitable for use in the present invention depending on the embodiment.

Non-limiting examples of taste-masking agents include cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat.®., Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel.®., Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IRO; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other embodiments, additional taste-masking materials contemplated are those described in U.S. Pat. Nos. 4,851,226, 5,075,114, and 5,876,759, each of which is hereby incorporated by reference in its entirety.

Non-limiting examples of pH modifiers include sodium carbonate or sodium bicarbonate.

### EXAMPLES

The following examples illustrate various aspects of the invention.

### Example 1

### In Vitro Assessment of Peroxynitrite-Decomposition Activity

To assess the peroxynitrate decomposition activity of the metalloporphyin-based PNDC compounds, the following experiments were conducted. An in vitro assay based on the inhibition of the peroxynitrite-mediated oxidation of aryl boronic was used to assess the activity of four metalloporphyin-based PNDC compounds: TCP-1, TCP-19, and TCP-4 with (R=H) from Table 2, and the octahydroacridine bis-salicylimine derivative illustrated in Formula 5 above, with no R substitution.

Stock solutions of 4-nitrophenylboronic acid, 4-acetylphenylboronic acid and the PNDCs were prepared in DMSO at concentrations of 5-50 mM. Peroxynitrite in 0.1 N NaOH solution was prepared by the method of Pryor and frozen at −80° C. until needed. Small aliquots of the PN solution were thawed, kept on ice and the concentration of each aliquot was measured by UV spectroscopy just before measurements were made. Peroxynitrite concentrations ranged from 58-77 mM prior to making any measurements. In a typical procedure 9.5×10⁻⁷ moles of 4-nitrophenylboronic acid (24.0 μL of stock) was dispensed into a small vial equipped with a magnetic stir bar. 1.00 mL of 250 mM phosphate buffer (pH=7.2) which contained 0.7% sodium dodecyl sulphate and 100 μM DTPA was added followed by 9.5×10⁻⁷ moles of the PNDC (aliquot from DMSO stock). To this rapidly stirred mixture was added 9.5×10⁻⁷ moles peroxynitrite by rapid injection. The mixture was stirred for one minute and analyzed by LCMS (Waters Alliance-MS3100 system; 15% acetonitrile/H2O to 95% acetonitrile (0.05% TFA) over 10 minutes; Agilent Eclipse XD8-C18 column, 5 μM, 4.6×150 mm, UV detection at 320 nm for 4-nitrophenol and 280 nm for 4-hydroxyacetophenone oxidation products). Reactions were run in triplicate and compared to controls (also run in triplicate) which contained everything except the PNDC (amounts of DMSO which were equivalent to those from aliquoted PNDC solutions were added to the controls to compensate for the very small effect of DMSO). The peak areas for phenol oxidation products were compared for catalyst versus control runs to determine percent inhibition. As a positive control for inhibition and a benchmark for estimating second order rate constants, Ebselen, an existing compound, was also run in this assay. The results of this assay are summarized in Table 8.

TABLE 8
Inhibition of PN-mediated Arylboronate Oxidation by PNDCs

| | | | | Estimated kₐ |
|---|---|---|---|---|
| | | % Inhibition | | (known k) |
| No. | Structure | PNB | APB | M⁻¹s⁻¹ |
| TCP-1 | 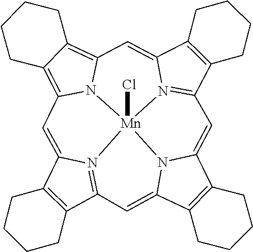 | 15.4 ± 2.5 | — | 1 × 10⁵ |
| TCP-19 | 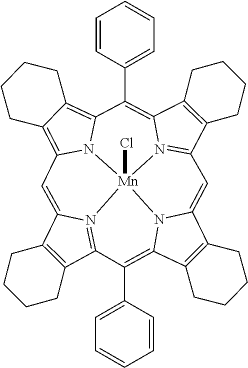 | 22.3 ± 0.8 | 11.5 ± 0.4 | 2 × 10⁵ |
| TCP-4 | 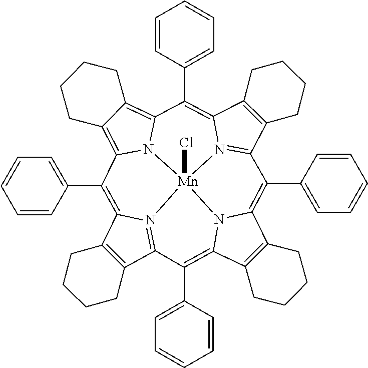 | 14.6 ± 1.7 | 9.87 ± 1.16 | 1 × 10⁵ |
| OBS from Formula (VI) | 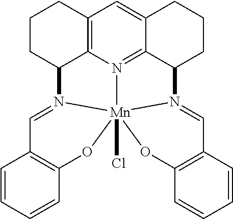 | 5.41 ± 1.5 | — | 1 × 10⁴ |
| Ebselen | — | 51.7 ± 1.1 | — | 1.7 × 10⁶ |
| | | | | (2 × 10⁶) |

### Example 2

### In Vitro Assessment of PNDC Transport Across Lipid Bilayers

To assess the lipophilicity and membrane transport properties of the metalloporphyin-based PNDC compounds, the following experiments were conducted. In vitro measurements of partition coefficients (log P, log D) were conducted in the presence and absence of biologically relevant ions that may coordinate the metal center of the compounds and further facilitate or hinder membrane transport.

A slow-stir method was used to assess log P for three molecules described previously in Table 2 above, and listed in Table 9 below: TCP-1, TCP-19, and TCP-6. Octanol and ultrapure water were pre-equilibrated for 24 hours before the slow-stir experiments were performed. A solution of 2-5 mg of each of the PNDC compounds in pre-equilibrated octanol (2-5 mL) was prepared. For each run, this octanol solution was carefully added to about 5 mL of pre-equilibrated ultrapure water. The solutions were stirred at a speed of 60 rpm and a temperature of 25° C. After stirring for 48 hours, the solutions were analyzed by HPLC. Log P was determined using the relation:

$$log [ \frac{concentration\_in \_oc tan ol}{cooncentration\_in \_water} ] = log [ \frac{{( peak\_area \cdot dilution\_factor )}_{oc tan ol}}{{( peak\_area \cdot dilution\_factor )}_{water}} ]$$

Log p values determined for the PNDCs are presented in Table 9:

TABLE 9
LogP for PNDC Compounds

| No. | Structure | logP |
|---|---|---|
| TCP-1 | 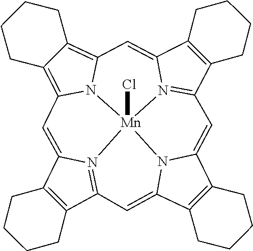 | 3.77 |
| TCP-19 | 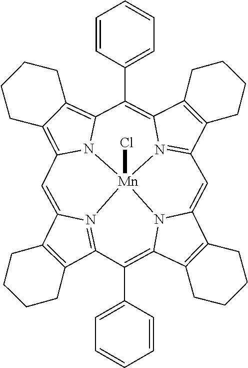 | 2.78 |
| TCP-9 | 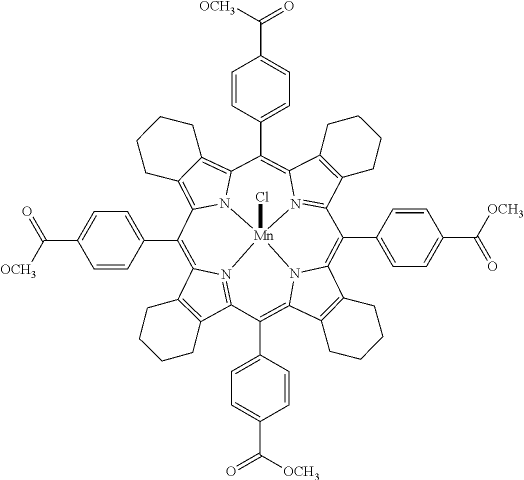 | 4.46 ± 0.19 |

The log P values for all compounds in Table 9 are consistent with high membrane solubility as compared to existing highly charged PNDC compounds such as Mn(III)TMPyP5+, which has a reported log P of −4.49.

### Example 3

### Assessment of Efficacy of Injected PNDC Compounds In Vitro

To assess the in vitro efficacy of a PNDC compound similar to those described above, the following experiments were conducted. The inhibition of LPS-stimulated TNF-α production by BV2 microglia cultures was assessed for cultures treated with the OP-11 compound described in Table 7 above at concentrations ranging from 1 μM to 30 μM.

BV2 microglia cell cultures were passaged twice a week and maintained in DMEM supplemented with 5% FBS, 50 IU/mL penicillin, and 50 pg/mL streptomycin at 37° C. in an atmosphere containing 5% CO₂ and 95% humidity. For the experiment, the BV2 cultures were plated overnight in 96-well cell-culture plates at 8×10⁴ cells/well in supplemented DMEM. The overnight cultures were treated with 1-30 μM OP-11 compound solutions or DMSO vehicle for 60 min in fresh supplemented DMEM prior to 3 hr stimulation of the culture with 30 ng/mL of LPS. Cell supernatant was drawn from each culture after 3 hr of LPS stimulation.

The BV2 cell supernatant was clarified by centrifuging for 5 min at 800 g and frozen at −80° C. The cell monolayers were lysed with isovolumetric ice-cold lysis buffer (20 mM Tris-Cl, pH 7.4, 100 mM NaCl, 1% Triton X-100, 50 mM NaF, 1 mM Na3VO4, 10 μg/mL Leupeptin, and 10 μg/mL Aprotinin) and clarified by centrifugation at 13,000 g for 10 min at 4° C. and stored at −80° C. The LPS-stimulated TNF-α expression was measured in the supernatant fractions by a horseradish peroxidase-conjugated sandwich ELISA using paired anti-murine TNF-α antibodies. In this ELISA, undiluted BV2 cell culture supernatant was thawed and incubated for 1 hr at 37° C. in 96-well plates previously coated with a primary anti-murine TNF-α antibody (1.2 μg/mL) and blocked with 1% BSA. The plates were then washed in 1× PBS with 0.05% Tween-20. The bound TNF-α on the plates was then visualized using HRPO-conjugated secondary antibody (300 ng/mL) and TMB reagent. The TNF-α concentration in each well of the plate was determined against calibrated standards developed using similar measurements using 0-2000 μg/mL concentrations of recombinant murine TNF-α in supplemented DMEM. The measured TNF-α concentrations were normalized against the total protein concentration of the cell lysate as determined by bicinchoninic acid assay.

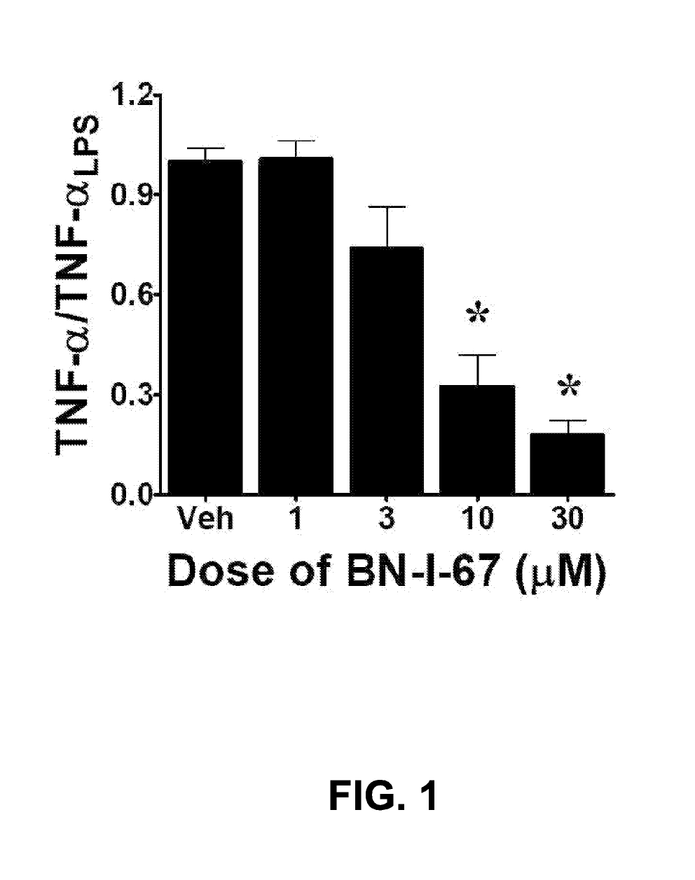

FIG. 1 summarizes the measured inhibition of TNF-α production by the BV2 cell cultures 3 hrs after LPS stimulation as a function of the concentration of OP-11 treatment. The increase in LPS-stimulated TNF-α production was inhibited by OP-11 treatment in a dose-dependent manner, with significant reductions in LPS-stimulated TNF-α production due to OP-11 treatment at concentrations of at least 10 μM. The in vitro IC₅₀ for the inhibition of LPS-stimulated TNF-α production by OP-11 treatment was determined to be about 3.5 μM.

### Example 4

### Assessment of Efficacy of Injected PNDC Compounds

To assess the efficacy of several injected PNDC compounds similar to those described above, the following experiments were conducted. The inhibition of carrageenan-induced thermal hyperalgesia in rats was assessed after subplantar injection of five different PNDC compounds, listed in Table 10 below.

TABLE 10
PNDC Compounds Injected Prior to Carrageenan Treatment

| | | Injected |
|---|---|---|
| | | Concentration |
| No. | Structure | (μM) |
| TCP-1 | 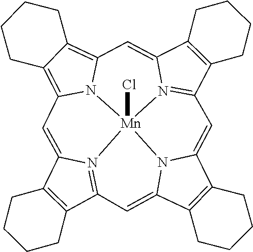 | 100 |
| TCP-19 | 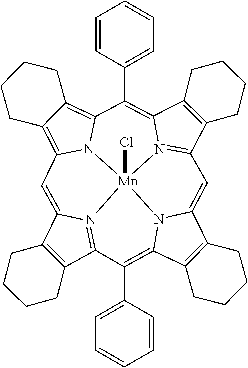 | 100 |
| OP-10 | 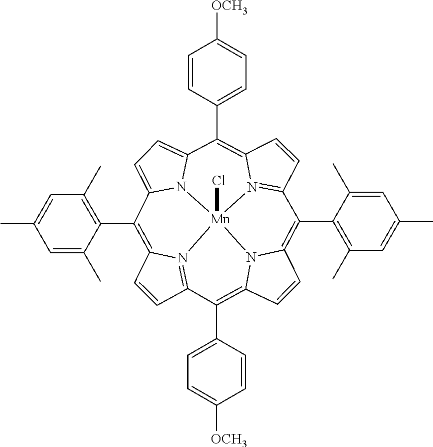 | 100 |
| OP-11 | 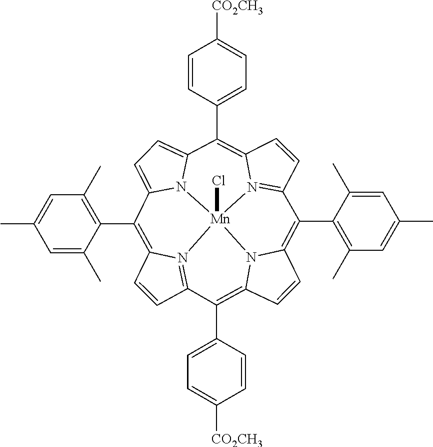 | 30, 100 |
| TCP-9 | 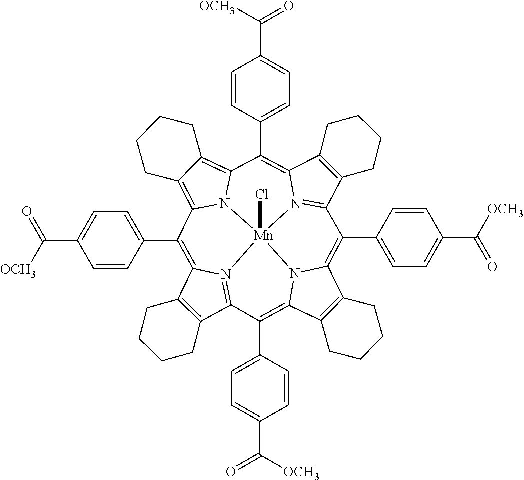 | 30 |

Lightly anesthetized rats [CO₂ (80%)/O₂ (20%)] received a subplantar injection of carrageenan (50 μL of a 1% by weight suspension of carrageenan in 0.85% NaCl solution) into the right hindpaw. Hyperalgesic responses of the injected rats to heat then were determined at specified time points (Hargreaves et al. 1988) using a cutoff latency of 20 s to prevent tissue damage. Rats were individually confined to plexiglass chambers, and a mobile unit consisting of a high intensity projector bulb was positioned to deliver a thermal stimulus directly to an individual hindpaw from beneath the chamber. The withdrawal latency periods for the injected paws were determined to the nearest 0.1 s with an electronic clock circuit and thermocouple.

The compounds listed in Table 10 were given by intraplantar injection 30 min before the intraplantar injection of carrageenan in non-fasted rats. As summarized in Table 10, 30 mL of OP-11 was injected at a concentration of 30 μM and 100 μM, TCP-9 was injected at a concentration of 30 μM, and TCP-1, TCP-19, and OP-10 were all injected at a concentration of 100 μM.

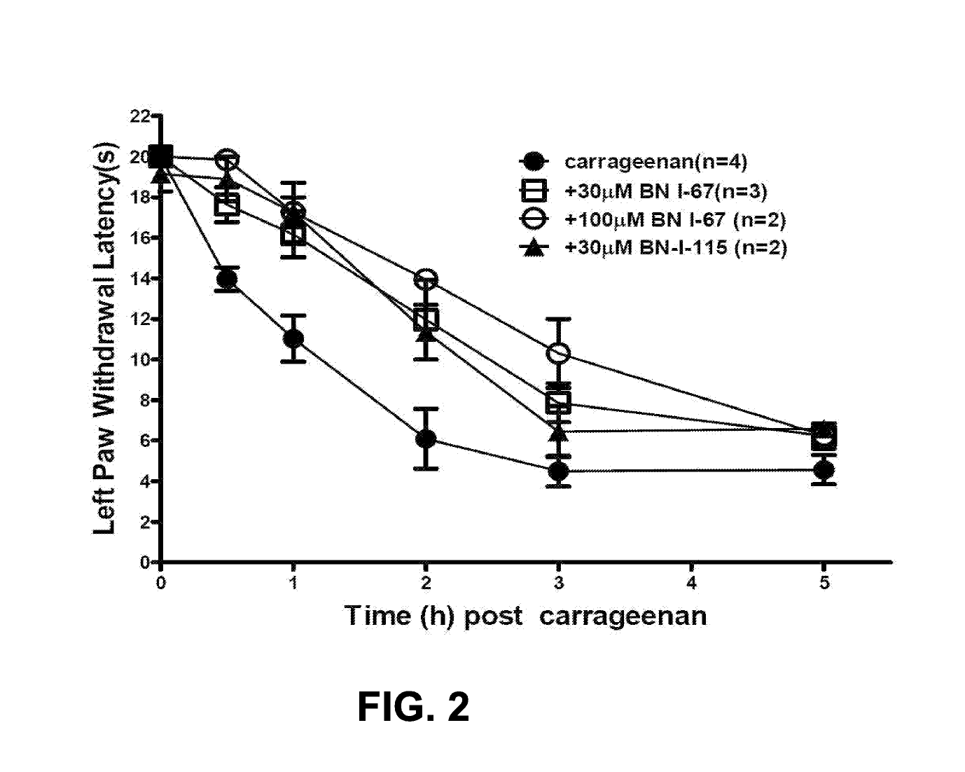

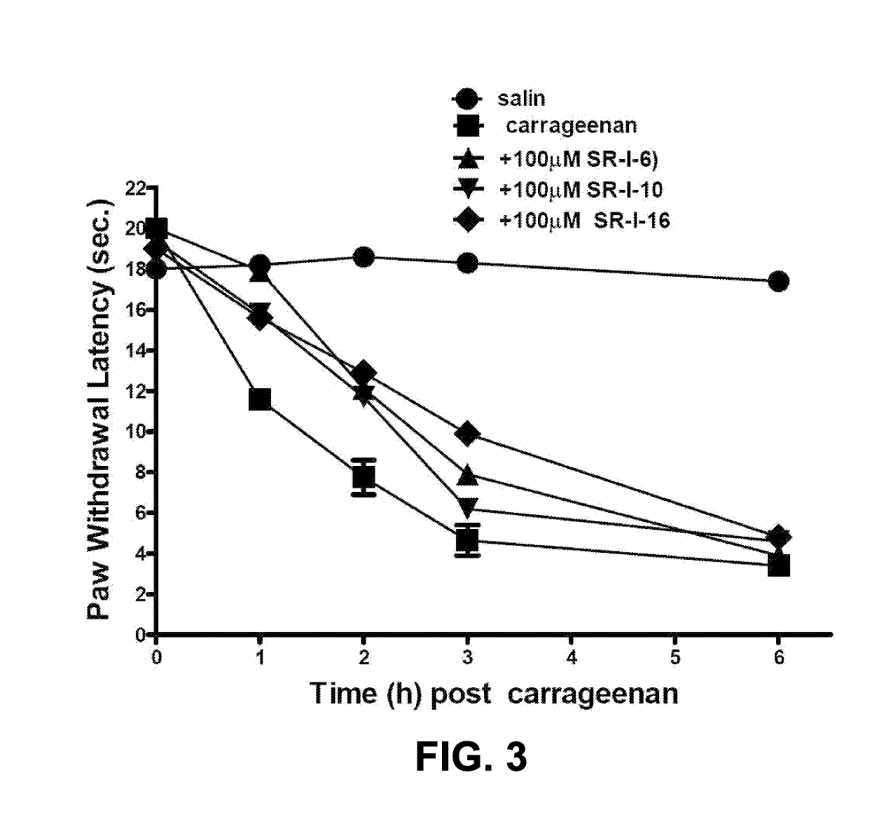

The results of these experiments are summarized in FIG. 2 and FIG. 3. The results in these figures are summarized in terms of changes at each time point in withdrawal latency, which were calculated as the difference in the withdrawal latencies of each rat at each different time points after carrageenan injection and the baseline withdrawal latencies measured prior to carrageenan injection.

As shown in FIG. 2, the interplantar injection of OP-11 inhibited the carrageenan-induced thermal hyperalgesia in a dose-dependant manner, and the interplantar injection of TCP-9 similarly inhibited the carrageenan-induced thermal hyperalgesia. Interplantar injection of TCP-1, TCP-19, and OP-10 similarly inhibited the carrageenan-induced thermal hyperalgesia of the rats, as shown in FIG. 3.

### Example 5

### Assessment of Efficacy of Orally Administered PNDC Compounds

To assess the efficacy of several orally administered PNDC compounds similar to those described above, the following experiments were conducted. The inhibition of carrageenan-induced thermal hyperalgesia in rats was assessed after oral administration of two different PNDC compounds, listed in Table 11 below:

TABLE 11
PNDC Compounds Injected Prior to Carrageenan Treatment

| | | Orally |
|---|---|---|
| | | Administered |
| | | Concentration |
| No. | Structure | (mpk) |
| TCP-1 | 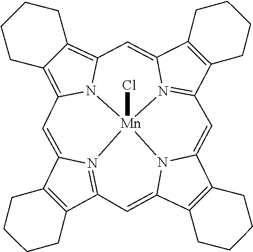 | 100 |
| TCP-19 | 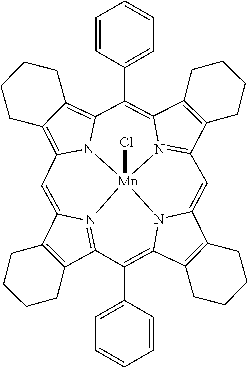 | 100 |
| TCP-4 | 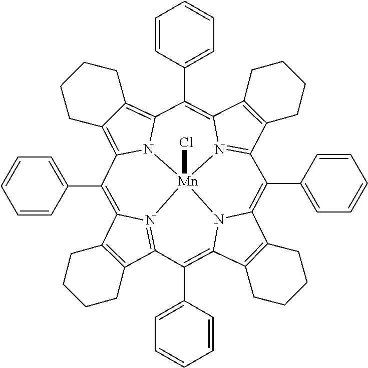 | 100 μM (injected) |

Thermal hyperalgesia was induced in rats by the interplantar injection of carrageenan using methods similar to those described in Example 4. The TCP-1 and TCP-10 compounds were given to the rats by oral gavage thirty minutes prior to carrageenan injection at 100 mg/kg dosages. For comparison, subplantar injections of TCP-7 were administered at a concentration of 100 μM using methods similar to those described in Example 4.

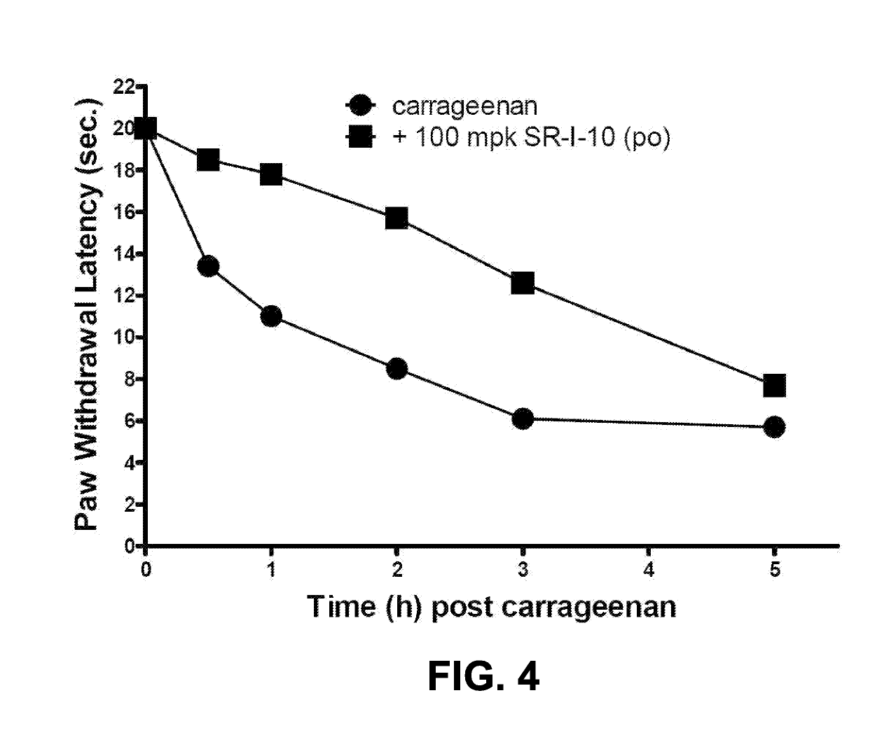

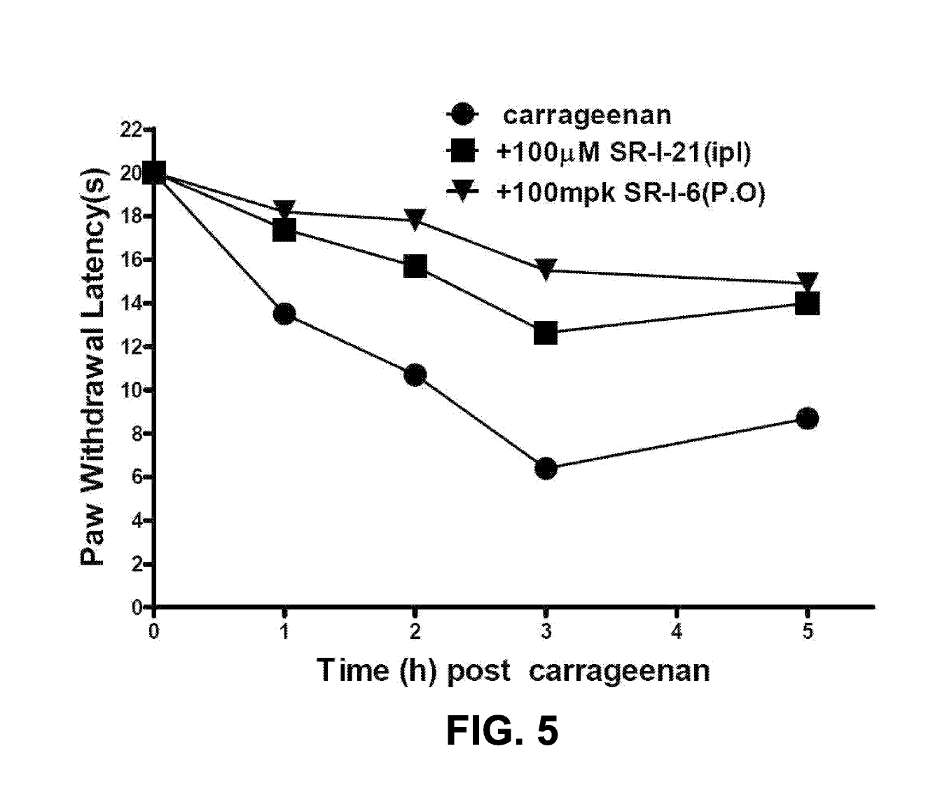

The carrageenan-induced thermal hyperalgesia was assessed in the rats using methods similar to those described in Example 4. The results of these experiments are summarized in FIG. 4 and FIG. 5. As shown in FIG. 4, the oral administration of TCP-19 inhibited the carrageenan-induced thermal hyperalgesia in the rats. The oral administration of TCP-1 similarly inhibited the carrageenan-induced thermal hyperalgesia of the rats, and was slightly more effective then an interplantar injection of TCP-4, as shown in FIG. 5.

### Example 6

### Assessment of Efficacy of Orally Administered PNDC Compound for Taxol-Induced Hyperalgesia

To assess the efficacy of an orally administered PNDC compound at inhibiting taxol-induced thermal hyperalgesia, the following experiments were conducted.

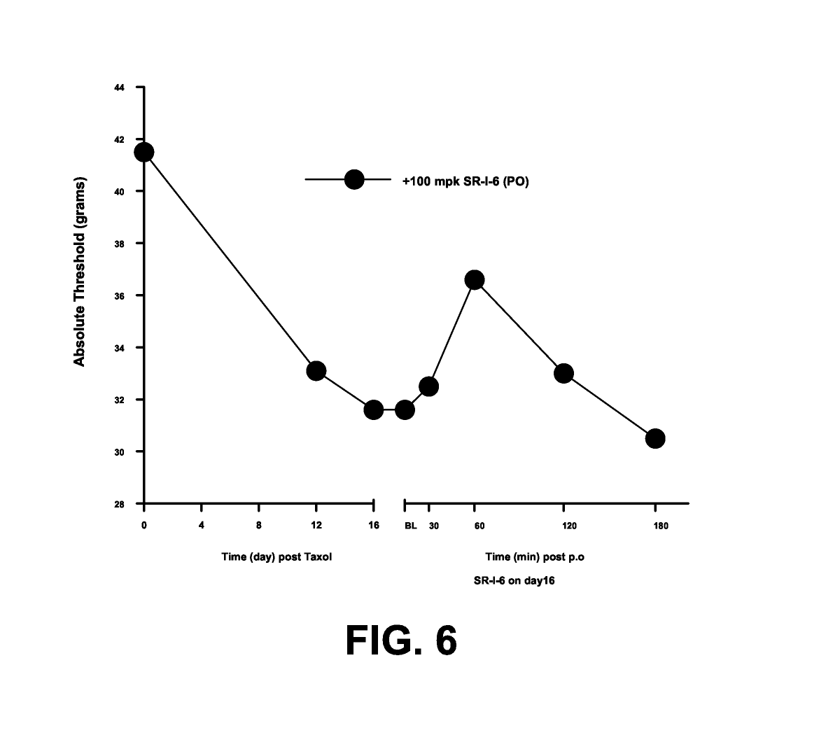

Thermal hyperalgesia was induced in rats by administering taxol at 1 mg/kg per dose on days 0, 2, 4, and 6 of a 6-day regimen. The inhibition of the taxol-induced hyperalgesia was assessed after the oral administration of TCP-1 by oral gavage at a dosage of 100 mpk, using methods similar to those described in Example 4. The results of this experiment are summarized in FIG. 6.

As shown in FIG. 6, the oral administration of TCP-1 at a dosage of 100 mpk reduced the taxol-induced thermal hyperalgesia for a period of about an hour after administration.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

All publications, patents, patent applications and other references cited in this application are herein incorporated by reference in their entirety as if each individual publication, patent, patent application or other reference were specifically and individually indicated to be incorporated by reference.

What is claimed is:

1. A method of treating a chronic pain disorder comprising administering a pharmaceutical PNDC compound comprising one or more tetracyclohexanoporphyrins comprising at least one transition metal ion.

2. The method of claim 1, wherein the pharmaceutical PNDC compound is administered orally as an oral composition.

3. The method of claim 2, wherein the oral composition comprises the pharmaceutical PNDC compound and further comprises at least one excipient chosen from binders, fillers, non-effervescent disintegrants, effervescent disintegrants, preservatives, diluents, flavor-modifying agents, sweeteners, lubricants, dispersants, color additives, taste-masking agents, pH modifiers, and combinations thereof.

4. The method of claim 3, wherein the oral composition is in a form chosen from tablets, chewable tablets, effervescent tablets, caplets, pills, powders, hard capsules, soft capsules, lozenges, sachets, sprinkles, reconstitutable powders, reconstitutable shakes, troches, pellets, granules, liquids, suspensions, emulsions, semisolids, and gels.

5. The method of claim 4, wherein the chronic pain disorder is selected from neurogenic pain and inflammatory pain.

6. The method of claim 5, wherein the pharmaceutical PNDC compound may be coadministered with one or more additional analgesic compounds comprising cyclooxygenase 2 inhibitors, non-selective NSAIDs, opiates and anti-metabolites, wherein the pharmaceutical PNDC compound and the one or more additional analgesic compounds exert a synergistic effect on the pain disorder.
